(19) United States
(12) Patent Application Publication
(10) Pub. No.: US 2004/0081969 A1
(43) Pub. Date: Apr. 29, 2004

(54) Devices and methods for evaulating the quality of a sample for use in an array assay

(76) Inventors: Diane D. Ilsley, San Jose, CA (US); Douglas A. Amorese, Los Altos, CA (US); Karen W. Shannon, Los Gatos, CA (US); Paul K. Wolber, Los Altos, CA (US)

Correspondence Address: Gordon Stewart, Agilent Technologies, Inc., Legal Dept., DL429, Intellectual Property Administration, P.O. Box 7599, Loveland, CO, 80537-0599, US

(21) Appl. No.: 10/283,785

(22) Filed: Oct. 29, 2002

(51) Int. Cl. C12Q 1/68; G01N 33/53; G01N 33/542; G06F 19/00; G01N 33/48; G01N 33/50

(52) U.S. Cl. 435/6; 435/7.9; 702/20

(57) ABSTRACT

Methods for evaluating whether a sample of at least one detectably-labeled target biomolecule is suitable for use in an array based assay are provided. The subject methods include: (1) providing a substrate having an evaluation array thereon; (2) contacting the evaluation array with the sample; (3) detecting any resultant target biomolecules to obtain signal data; and (4) processing the signal data to evaluate whether the sample is suitable for use in a biopolymeric array assay. Many embodiments of the subject methods include using a volume of sample that does not exceed about 5 μl and/or incubating the sample with the array for a period of time that does not exceed about 4 hours. Also provided are methods of performing array based assays that include the subject evaluation methods, and kits for practicing the subject methods.

### FIELD OF THE INVENTION

The field of this invention is biopolymeric arrays.

### BACKGROUND OF THE INVENTION

Array assays between surface bound binding agents or probes and target molecules in solution may be used to detect the presence of particular analytes or biopolymers in the solution. The surface-bound probes may be oligonucleotides, peptides, polypeptides, proteins, antibodies or other molecules capable of binding with target biomolecules in the solution. Such binding interactions are the basis for many of the methods and devices used in a variety of different fields, e.g., genomics (in sequencing by hybridization, SNP detection, differential gene expression analysis, identification of novel genes, gene mapping, finger printing, etc.) and proteomics.

One typical array assay method involves biopolymeric probes immobilized in an array on a substrate such as a glass substrate or the like. A solution containing target molecules (“targets”) that bind with the attached probes is placed in contact with the bound probes under conditions sufficient to promote binding of targets in the solution to the complementary probes on the substrate to form a binding complex that is bound to the surface of the substrate. The pattern of binding by target molecules to probe features or spots on the substrate produces a pattern, i.e., a binding complex pattern, on the surface of the substrate which is detected. This detection of binding complexes provides desired information about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on t h e array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

Accordingly, to detect such binding complexes in array assays, the targets in solution are labeled with a detectable label or tag such as a fluorescent label, chemiluminescent label, radioactive label or the like. A variety of methods are known for preparing the labeled targets. For example, where the targets are fluorescently labeled representations of mRNA populations, reverse transcription of mRNA using an oligo-dT primer that binds to the polyA tail of mRNA may be employed and detectably labeled nucleotides are incorporated into the cDNA during synthesis. As such, for each mRNA molecule, detectably-labeled cDNA target is produced. Another method employs linear mRNA amplification to generate detectably labeled antisense RNA. In this method, mRNA is converted to a double-stranded cDNA intermediate using oligo-dT primer linked to a promoter sequence. RNA polymerase then recognizes the promoter sequence in the cDNA and incorporates a detectable label into the antisense RNA transcribed therefrom. Multiple copies may then be transcribed from each cDNA.

It is apparent that the quality of results of such array binding assays is directly related to the quality of the labeled sample that is contacted with the bound probes. For example, signal quality may be compromised when the labels bound to targets provide poor or low signal and/or the labels have not been incorporated into the target biomolecule such that “free” labels, i.e., labels not attached to targets, are present in the sample, as well as unlabeled targets. Both cases may result in bound target biomolecules escaping detection and, in the latter case, unincorporated labels may give rise to elevated background signal, all of which may compromise the array assay results.

One problem with target preparation, regardless of how the target is prepared, is the lack of an effective, easy, cost-effective method to validate or evaluate the labeled targets prior to use in an array assay such as a hybridization assay or the like. One method that is currently used employs UV spectrophotometry to determine the amount of nucleic acid generated and the amount of detectably labeled nucleotide incorporated. Another method commonly used employs gel electrophoresis to visualize the targets and determine the size distribution thereof. However, both of these methods are indirect measurements of the targets in that they do not necessarily predict how the targets will perform when used in an array assay such as a hybridization assay. For example, unincorporated nucleotides and unincorporated labeled nucleotides can elevate the UV measurement, thereby providing a false indication that the target labeling process was successful and also resulting in elevated background signal on an array when the sample is contacted with an array. Furthermore, depending on the target preparation method, the amount of target generated may be very small, such that the fraction available for validation is below the sensitivity of detection for UV spectrophotometry and gel electrophoresis. It is apparent that a large enough or sufficient amount of sample must be left over from the sample evaluation for use in a subsequent array assay using the target.

Accordingly, there continues to be an interest in the development of new methods and devices for evaluating the quality of a sample of detectably labeled target biomolecules prior to use in an array assay. Of particular interest is the development of such methods and devices that are easy to use, cost effective, can be performed in a short period of time, require a small sample amount and that may also be capable of evaluating multiple samples at the same time without cross-contamination.

### SUMMARY OF THE INVENTION

Methods for evaluating whether a sample of at least one detectably-labeled target biomolecule is suitable for use in an array based assay are provided. The subject methods include: (1) providing a substrate having an evaluation array thereon; (2) contacting the evaluation array with the sample; (3) detecting any resultant bound target biomolecules to obtain signal data; and (4) processing the signal data to evaluate whether the sample is suitable for use in a biopolymeric array assay. Many embodiments of the subject methods include using a volume of sample that does not exceed about 5 μl and/or incubating the sample with the array for a period of time that does not exceed about 4 hours. Also provided are methods of performing array based assays that include the subject evaluation methods, and kits for practicing the subject methods.

### BRIEF DESCRIPTIONS OF THE DRAWINGS

FIG. 1 shows an exemplary embodiment of a sample evaluation device according to the subject invention that includes a substrate carrying a sample evaluation array for evaluating the quality of a sample having at least one detectably labeled target biomolecule.

FIG. 2 shows an enlarged view of a portion of FIG. 1 showing spots or features.

FIG. 3A shows an exemplary embodiment of a sample evaluation device according to the subject invention including a substrate carrying a plurality of sample evaluation arrays. FIG. 3B shows an enlarged view of one of the sample evaluation arrays of the device of FIG. 3A showing spots or features.

FIG. 4 shows the results of using a subject device to evaluate the quality of a sample for use in an array assay.

FIG. 5 shows the results of using a subject device to evaluate the quality of a sample for use in an array assay wherein the gene of interest is not expressed in the sample.

FIG. 6 shows the results of using a subject device to evaluate the quality of a sample for use in an array assay wherein the sample exhibits cross hybridization and non-specific binding.

FIG. 7 shows the results of using a subject device to evaluate the quality of two samples for use in an array assay wherein the quality of one of the sample is determined to be suitable for use in an array assay and the quality of the other sample is determined not to be suitable for use in an array assay.

FIG. 8 shows the log ratio of the results of FIG. 8.

### DEFINITIONS

The term “polymer” refers to any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

The term Amonomer@ as used herein refers to a chemical entity that can be covalently linked to one or more other such entities to form an oligomer. Examples of Amonomers@ include nucleotides, amino acids, saccharides, peptides, and the like. In general, the monomers used in conjunction with the present invention have first and second sites (e.g., C-termini and N-termini, or 5′ and 3′ sites) suitable for binding to other like monomers by means of standard chemical reactions (e.g., condensation, nucleophilic displacement of a leaving group, or the like), and a diverse element which distinguishes a particular monomer from a different monomer of the same type (e.g., an amino acid side chain, a nucleotide base, etc.). The initial substrate-bound monomer is generally used as a building-block in a multi-step synthesis procedure to form a complete ligand, such as in the synthesis of oligonucleotides, oligopeptides, and the like.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure.

The term “probe” used herein refers to a moiety that is capable of covalently or otherwise chemically binding a compound or molecule, of interest. The term Aprobe@ in the context of the invention may or may not be an Aoligomer@ as defined above. The term Aprobe@ as used herein may also refer to a compound that is synthesized on a substrate surface as well as a compound that is Apre-synthesized@ or obtained commercially, and then attached to a substrate surface.

The terms “array” “biopolymeric array” and “microarray” are used herein interchangeably to refer to an arrangement of probes or polymeric binding agents stably attached to a substrate surface which can be used for sample evaluation, analyte detection, combinatorial chemistry, or other applications wherein a two-dimensional arrangement of molecules of interest can be used. That is, the terms refer to an ordered pattern of probe molecules adherent to a substrate, i.e., wherein a plurality of molecular probes are bound to a substrate surface and arranged in a spatially defined and physically addressable manner. The probes or polymeric binding agents may vary widely, however polymeric binding agents of particular interest include peptides, proteins, nucleic acids, polysaccharides, synthetic mimetics of such biopolymeric binding agents, etc.

In many embodiments of interest, the biopolymeric arrays are arrays of nucleic acids, including oligonucleotides, polynucleotides, cDNAs, mRNAs, cRNAs, synthetic mimetics thereof, and the like. Such arrays may be comprised of oligonucleotides, peptides, polypeptides, proteins, antibodies, or other molecules used to detect target molecules in a sample.

The term “biomolecule” means any organic or biochemical molecule, group or species. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g. deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in hybridization reactions, i.e., cooperative interactions through Pi electrons stacking and hydrogen bonds, such as Watson-Crick base pairing interactions, Wobble interactions, etc.

The terms “ribonucleic acid” and “RNA”s used herein mean a polymer composed of ribonucleotides.

The terns “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

The term “sample” as used herein relates to a material or mixture of materials, typically, although not necessarily, in fluid form, containing one or more target biomolecules.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “communicating” information refers to transmitting data representing that information as electrical signals over a suitable communication channel (for example, a private or public network).

The term “forwarding” an item refers to any means of getting that item from one location to the next, whether by physically transporting that item or otherwise (where that is possible) and includes, at least in the case of data, physically transporting a medium carrying the data or communicating the data.

The terms “target” “target molecule” “target biomolecule” and “analyte” are used herein interchangeably and refer to a known or unknown molecule in a sample, which will bind, e.g., hybridize, to a probe on a substrate surface if the target molecule and the molecular probe contain complementary regions, i.e., if they are members of a specific binding pair. In general, the target molecule is a biopolymer, i.e., an oligomer or polymer such as an oligonucleotide, a peptide, a polypeptide, a protein, nucleic acid, and antibody, or the like.

As used herein, the terms “reporter,” “label” “detectable reporter” and “detectable label” refer to a molecule capable of generating a measurable signal, including, but not limited to, radioactive isotopes, fluorescers, chemiluminescers, enzymes, enzyme substrates, enzyme cofactors, enzyme inhibitors, dyes, metal ions, metal sols, ligands (e.g., biotin or haptens) and the like. The term “fluorescer” refers to a substance or a portion thereof which is capable of exhibiting fluorescence in the detectable range when excited at the appropriate wavelength. The term “cofactor” is used broadly herein to include any molecular moiety which participates in an enzymatic reaction. Particular examples of labels which may be used under the invention include fluorescein, 5(6)-carboxyfluorescein, Cyanine 3 (Cy3), Cyanine 5 (Cy5), rhodamine, dansyl, umbelliferone, Texas red, luminol, NADPH, α,β-galactosidase and horseradish peroxidase.

The terms “feature” and “spot” as used herein refer to a set of plurality of probes, wherein the probes are bound to a surface. Usually, the probes are bound to a surface such that each set of probes is arranged in a spaced-apart relation to each other at known locations. That is, a feature is the region of the array that contains probes, where the features may be separated by regions devoid of probes, and each feature occurs at approximately known locations and is distinct from other features.

The term “hybridization” as used herein refers to binding between complementary or partially complementary molecules, for example as between the sense and anti-sense strands of double-stranded DNA. Such binding is commonly non-covalent binding, and is specific enough that such binding may be used to differentiate between highly complementary molecules and others less complementary. Examples of highly complementary molecules include complementary oligonucleotides, DNA, RNA, and the like, which comprise a region of nucleotides arranged in the nucleotide sequence that is exactly complementary to a probe; examples of less complementary oligonucleotides include ones with nucleotide sequences comprising one or more nucleotides not in the sequence exactly complementary to a probe oligonucleotide.

The term “hybridization solution” or “hybridization reagent” used herein interchangeably refers to a solution suitable for use in a hybridization reaction.

The term “remote location” refers to a location other than the location at which the array is present and hybridization occur. As such, when one item is indicated as being “remote” from another, what is meant is that the two items are at least in different buildings, and may be at least one mile, ten miles, or at least one hundred miles apart.

The term “substrate” as used herein refers to a surface upon which probes may be adhered to provide an array. Glass slides are the most common substrate for arrays, although fused silica, silicon, plastic and other materials are also suitable.

The term “stringent hybridization conditions” as used herein refers to conditions that are that are compatible to produce duplexes on an array surface between complementary binding members, i.e., between probes and complementary targets in a sample, e.g., duplexes of nucleic acid probes, such as DNA probes, and their corresponding nucleic acid targets that are present in the sample, e.g., their corresponding mRNA analytes present in the sample. An example of stringent hybridization conditions is hybridization at 60° C. or higher and 3×SSC (450 mM sodium chloride/45 mM sodium citrate). Another example of stringent hybridization conditions is incubation at 42° C. in a solution containing 30% formamide, 1M NaCl, 0.5% sodium sarcosine, 50 mM MES, pH 6.5. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

### DETAILED DESCRIPTION OF THE INVENTION

Methods for evaluating whether a sample of at least one detectably-labeled target biomolecule is suitable for use in an array based assay are provided. The subject methods include: (1) providing a substrate having an evaluation array thereon; (2) contacting the evaluation array with the sample; (3) detecting any resultant bound target biomolecules to obtain signal data; and (4) processing the signal data to evaluate whether the sample is suitable for use in a biopolymeric array assay. Many embodiments of the subject methods include using a volume of sample that does not exceed about 5 μl and/or incubating the sample with the array for a period of time that does not exceed about 4 hours. Also provided are methods of performing array based assays that include the subject evaluation methods, and kits for practicing the subject methods.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, the subject invention provides methods for evaluating whether a sample of at least one detectably labeled target biomolecule is suitable for use in an array assay, i.e., suitable for use in an array binding assay. By array assay or array binding assay is meant any suitable binding assay in which members of a specific binding pair interact, e.g., for analyte detection, etc. That is, the subject invention provides methods for the evaluation of a sample of at least one target biomolecule, where the sample may be used in any of a number of different array assays where typically a first member of a binding pair, i.e., a probe, is stably associated with the surface of a substrate and a second member of a binding pair, i.e., a target, is free in the sample, where the binding members may be: ligands and receptors, antibodies and antigens, complementary nucleic acids, and the like. For ease of description only, the subject devices and methods as described below will oftentimes be described in reference to hybridization assays of nucleic acids, where such examples are not intended to limit the scope of the invention. It will be appreciated by those of skill in the art that the subject invention may be employed to evaluate the quality of a sample of targets for use in other array non-nucleic acid assays as well, such as immunoassays, proteomic assays, etc.

In further describing the subject invention, devices for evaluating whether a sample is suitable for use in an array assay are described first, followed by a detailed description of the subject methods of using the devices to evaluate a target sample and array based assays that include the subject evaluation methods as well as kits for use in practicing the subject methods.

Sample Evaluation Devices

The subject invention includes sample evaluation devices for evaluating whether a sample of at least one detectably-labeled target is suitable for use in an array assay. That is, the subject devices are employed to evaluate a labeled sample prior to the sample being used in an array assay. Generally, the sample evaluation devices of the subject invention include a plurality of probes (i.e., binding agents) deposited or synthesized directly (i.e., synthesized in situ) onto the surface of a substrate in the form of an “array” or pattern, such that it is a sample evaluation array, where the subject sample evaluation devices may include one or more sample evaluation arrays and one or more of the arrays may be the same or one or more may be different. In many embodiments, the subject devices include from about 10 to about 200 sample evaluation arrays on a single sample evaluation device. The sample evaluation devices are designed to assess the quality of a sample that contains labeled target, e.g., fluorescently labeled cRNA or cDNA targets that are representative of cellular mRNA from a cell or tissue.

As mentioned above, the oligonucleotide probes of the subject invention are stably attached to a substrate surface, where each probe is present as multiple copies of an oligonucleotide sequence in the form of a spot or feature. The oligonucleotide probes may be nucleic acid, e.g., RNA, DNA, or nucleic acid mimetics. Accordingly, the subject devices include a plurality of distinct oligonucleotide probe features or spots. The probes that make-up the probe features are complementary to sequences within a labeled sample, i.e., complementary to targets in a sample, and thus when the sample is contacted with the probes, the sequences in the sample bind to their complementary probes, as will be described in greater detail below.

The probe features may be any convenient shape, but will typically be circular, oval, elliptical, annular, or some other analogous shape, but may also be squared, rectangular, triangular, etc., where in certain embodiments the shape of a particular probe feature is dictated by the particular method employed to fabricate the array. The size of the subject probe features will vary. By way of example and not limitation, where the feature has an overall circular or analogous shape, the diameter of the feature will generally range from about 0.1 μm to about 10 mm, usually from about 1 μm to about 500 μm and more usually from about 10 μm to about 200 μm. Features that are not circular may have equivalent dimensions.

The oligonucleotide probes present that make-up a subject evaluation array correspond to one or more genes that are expressed in a majority of tissues, cell types and species, i.e., one or more genes that are common to or are conserved in a great number of tissues, cell types and species, where typically such genes are those that are expressed at medium to high abundance within the cell, e.g., about 20 to about 200 messages/cell for medium expression and about 200 to about 3000 or more messages/cell for high expression. In this manner, the same array may be used to evaluate a number of different samples as the evaluation arrays represent genes common to a variety of cells and tissues. As such, each oligonucleotide feature on the array, excluding control features, calibration features, background features, etc., if present, will correspond to a gene that is common in most tissues and cell types.

The number of different genes represented in an array of the subject invention may vary, where in certain embodiments the number of different genes represented in the array will range from about 2 to about 100, usually from about 5 to about 50, where in certain embodiments the number of genes may be as high as about 100 or even about 200 or more. A gene is considered to be represented on an array if a target cRNA or cDNA derived from the gene is able to hybridize to at least one probe feature on the array. Genes that may be represented on the arrays of the subject invention include, but are not limited to: protein phosphatase. Ef-1 alpha, G3PDH, beta globin, and the like.

The total number of probes present that make-up a sample evaluation array will vary depending on a variety of factors such as, but not limited to, the number of genes represented on an array, the length of a gene represented on an array, the length of the overlapping regions of the probes that correspond to a gene, etc. Typically, about 2 to about 10 probes correspond to each gene such that about 4 to about 1000, usually about 10 to about 500 distinct features or probe spots are present as an array if the above-described number of genes is represented, where the number may be higher in certain embodiments. In many embodiments, it may be desirable to have each distinct probe feature present in duplicate, triplicate, etc., e.g., in some instances two features are present for each distinct probe sequence or even three or more features are present for each distinct probe sequence.

In many instances, the probes representing a gene are “tiled” across the gene from the 5′ to the 3′ end, and usually tiled across a gene such that an overlap in one or more bases exists between adjacent probes, i.e., between a probe and the next consecutive downstream probe. In this manner, a user is able to easily evaluate target synthesis, e.g., a user may determine the length of the targets, the binding specificity to the probes, etc. For example, for a particular gene, probes thereof may be about 60 bases in length and have about a 10 base overlap, where the probes may be produced by tiling or walking across the gene starting at the 5′ end. More specifically probes that make-up a sample evaluation array may include consecutively overlapping sequences of a gene such that a sequence, i.e., an overlapping sequence, is common to at least two probes. That is, as probes are generated from the 5′ end to the 3′ end, each two adjacent probes include a common sequence i.e., an overlapping sequence. For example, starting at the 5′ end of a gene and walking or tiling down the gene to the 3′ end, a first probe may represent bases 1-60 of the gene-typically multiple copies of such in the form of a first feature, a second probe may represent bases 50-110 of the gene-typically multiple copies of such in the form of a second feature, a third probe may represent bases 100-160 of the gene-typically multiple copies of such in the form of a third feature, etc., covering the entire coding sequence of the gene. Accordingly, adjacent probes include a common, overlapping sequence. That is, in this example, first and second probes each include overlapping bases 50-60 and second and third probes each include overlapping bases 100-110, and so on such that third and fourth probes each include overlapping bases 150-160, etc. Of course, the number of bases that overlap may vary such that fewer or greater than ten bases may make-up the overlapping sequence.

Similarly, probes may be designed that tile across relatively well-conserved genes between species such as human and mouse. These probes differ from the probes designed to a specific species in that they are intended to hybridize equally well to targets from a variety of species. Alignment of sequences has shown that in conserved genes, on average about every third base is degenerate (consistent with codon wobble positions). Therefore, for these probes, the location of the degenerate nucleotide may be designed to correspond to the third base of the codon.

While tiled probes may provide an assessment of the length of labeled target generated in a labeling process, probes may also be designed to provide a more global assessment of labeling efficiency. These global assessment type probes provide a great deal of information about the overall labeling efficiency of the targets, but not any one specific target. Typically, this is accomplished with a series of probes to repetitive sequences such as the Alu or Kpn repeats. These are present on the array in multiple copies, but distributed geographically across the array to avoid problems of target depletion. Since repeats are present in a large number of different targets, they are typically designed out of target specific probes, but are useful for this application.

Certain embodiments may include probes that are serially deleted from the 3′ end of a gene. For example, a probe set may include a 60 mer probe, a 50 mer probe, a 40 mer probe, a 30 mer probe, a 20 mer probe, and a 10 mer probe, all of which correspond to the same gene sequence, but which vary in length. For example, all the probes of such a probe set may include the same 3′ end, but vary with respect to the 5′ end because of their different lengths. In using such probes, sample binding, in relation to relative signal intensities, exhibits characteristic behavior that provides information about the sample. For example, when the results of contacting such a probe set with labeled sample is plotted on a graph having axes of signal intensity versus length, the signal intensity of bound labeled sample to these probes will correspond to a predictable curve such that the signal intensity is highest for the probe of longest length and decreases in a predictable manner for probes of successively shorter lengths. Furthermore, these probes may also serve to verify quality of the array fabrication process because of the reproducible curve generated by plotting signal intensity versus length.

The average length of an oligonucleotide probe on the array is sufficient to provide a strong and reproducible signal, as well as sufficient hybridization to its complementary target. In certain embodiments, the average length of the oligonucleotide probes typically ranges from about 10 nt to about 100 nt, usually from about 20 nt to about 80 nt and more usually from about 35 nt to about 70 nt. However, probes having shorter or greater lengths may be used also as appropriate.

The density of all the spots on the array substrate, i.e., both probe spots and non-probe spots, e.g., calibration spots, control spots, and the like, is at least about 1 spot/cm², and usually about 400 spots/cm², but typically does not exceed about 106 spots/cm², and in many embodiments does not exceed about 100,000 spots/cm², where in certain embodiments does not exceed about 1,000 spots/cm².

The amount of oligonucleotide present in each feature will be sufficient to provide for adequate hybridization and detection of labeled target during the assay in which the array is used. Generally, the amount of oligonucleotide in each feature will range from about 1,000 to about 100,000 molecules/feature, and more usually from about 5,000 to about 60,000 molecules/feature.

The spots may be arranged in any convenient pattern across or over the substrate surface, such as in the form of organized rows and columns of spots, e.g., a grid of spots, across the substrate surface, a series of curvilinear rows across the substrate surface, e.g., a series of concentric circles or semi-circles of spots, and the like. As mentioned above, usually more than one gene is represented on a subject array. As such, the subject sample evaluation devices may include a single pattern of spots or may include a plurality of different spot patterns, e.g., corresponding to different genes, such that the number of different spot patterns may be as great as the number of genes represented on the array, or as few as one single pattern of spots.

In certain embodiments, more than one array may be present on a single substrate, where one or more of the arrays present may be the same or different. In many embodiments, the same array, i.e., the same spot pattern or set of spot patterns, is repeated across the substrate surface to provide a plurality of the same sample evaluation arrays on a single substrate. In other words, the subject sample evaluation devices may include a plurality of arrays in which the same pattern of spots, i.e., the same probes, are reproduced in distinct positions on the substrate surface. As mentioned above, some or all of the arrays may be different. The number of arrays on a single substrate will vary, where the number may range from about 2 to about 200, where usually the number of arrays ranges from about 10 to about 200; however a greater number of arrays may be present in certain embodiments. In this manner, the subject devices enable the testing of a plurality of samples against the same array, i.e., the same set of probes, at the same time and under the exact same conditions without cross-contamination, or, in they case where one or more arrays may be different, the same sample (or different samples) may be tested against different arrays at the same time and under the exact same conditions without cross-contamination.

Where the devices include more than one array on a single substrate, each array is segregated from other arrays by appropriate physical, chemical and/or mechanical means so that each array is independent of any other arrays and sample is confined to a single array. As such, each array defines a respective array assay area. For example, the substrate may include walls or other physical barriers around an array. In certain embodiments, the surface of the substrate around each array may be modified to make it hydrophobic such that sample is retained within the hydrophobically defined array assay area around each array. Still further, because of the small sample volume required to use the sample evaluation arrays of the subject invention, typically the arrays are simply spaced-apart or spatially separated a suitable distance from each other such that substrate need not include barriers or walls, whether physical or chemical, to separate the arrays because the small size of the sample droplet applied to the array such that surface tension acting upon the droplet effectively confines the sample droplet to a single array. In this manner, manufacturing costs are minimized.

As mentioned above, the subject sample evaluation arrays usually include one or more additional features of oligonucleotides which do not correspond to the genes described above, i.e., they do not correspond to genes that are expressed in a majority of tissues, cell types and species. For example, positive and negative controls, calibration probes, background probes and the like may be present. For example one or more negative control probes, i.e., non-binding or non-evaluation probes, e.g., reverse complements of one or more of the sample evaluation probes, may be included, where such negative control probes do not possess any specific affinity for targets, e.g., do not possess specific affinity for target nucleic acid sequences and as such no target sequences should bind to these negative control probes. Binding detected at these negative control probes may be indicative of unbound label in the sample or non-specific binding or background from the sample. The sample devices may also include probes of housekeeping genes, for example human housekeeping genes such as protein phosphatase, EF-1 alpha, actin, and the like.

The sample evaluation arrays may be produced using any convenient protocol. Various methods for forming arrays from pre-formed probes, or methods for generating the array using synthesis techniques to produce the probes in situ, are generally known in the art. See, for example, Southern, U.S. Pat. No. 5,700,637; Pirrung, et al., U.S. Pat. No. 5,143,854 and Fodor, et al. (1991) Science 251:767-777, the disclosures of which are incorporated herein by reference and PCT International Publication No. WO 92/10092. For example, the suitable probes may either be synthesized directly on the solid support or substrate to be used in the array assay or attached to the substrate after they are made. Arrays may be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. Nos. 6,242,266, 6,232,072, 6,180,351, 6,171,797, and 6,323,043; and U.S. patent application Ser. No. 09/302,898 filed Apr. 30, 1999 by Caren et al., and the references cited therein, the disclosures of which are herein incorporated by reference. Other drop deposition methods may be used for fabrication. Also, instead of drop deposition methods, photolithographic array fabrication methods may be used such as described in U.S. Pat. Nos. 5,599,695, 5,753,788, and 6,329,143, the disclosures of which are herein incorporated by reference. As mentioned above, interfeature areas need not be present, particularly when the arrays are made by photolithographic methods as described in those patents.

As mentioned above, the sample evaluation arrays are present on a surface of a solid support. A variety of solid supports or substrates may be used, upon which a sample evaluation array may be stably associated. In certain embodiments, a plurality of arrays may be stably associated with one substrate. For example, a plurality of arrays may be stably associated with one substrate, where the arrays are spatially separated from some or all of the other arrays associated with the substrate. Where more than one array is present on a substrate surface, the arrays may be the same or different.

Substrates for use in the present invention may be selected from a wide variety of materials including, but not limited to, natural polymeric materials, particularly cellulosic materials and materials derived from cellulose, such as fiber containing papers, e.g., filter paper, chromatographic paper, etc., synthetic or modified naturally occurring polymers, such as nitrocellulose, cellulose acetate, poly (vinyl chloride), polyamides, polyacrylamide, polyacrylate, polymethacrylate, polyesters, polyolefins, polyethylene, polytetrafluoro-ethylene, polypropylene, poly (4-methylbutene), polystyrene, poly(ethylene terephthalate), nylon, poly(vinyl butyrate), cross linked dextran, agarose, etc.; either used by themselves or in conjunction with other materials; fused silica (e.g., glass), bioglass, silicon chips, ceramics, metals, and the like. For example, substrates may include polystyrene, to which short oligophosphodiesters, e.g., oligonucleotides ranging from about 5 to about 50 nucleotides in length, may readily be covalently attached (Letsinger et al. (1975) Nucl. Acids Res. 2:773-786), as well as polyacrylamide (Gait et al. (1982) Nucl. Acids Res. 10:6243-6254), silica (Caruthers et al. (1980) Tetrahedron Letters 21:719-722), and controlled-pore glass (Sproat et al. (1983) Tetrahedron Letters 24:5771-5774). Additionally, the substrate can be hydrophilic or capable of being rendered hydrophilic.

Suitable substrates may exist, for example, as sheets, tubing, spheres, containers, pads, slices, films, plates, slides, strips, disks, etc. The substrate is usually flat, but may take on alternative surface configurations. The substrate may be a flat, glass substrate, such as a conventional microscope glass slide, a cover slip and the like. Common substrates used for the sample evaluation arrays of probes are surface-derivatized glass or silica, or polymer membrane surfaces, as described in Maskos, U. et al., Nucleic Acids Res, 1992, 20:1679-84 and Southern, E. M. et al., Nucleic acids Res, 1994, 22:1368-73.

The size of the substrate will vary depending on a variety of factors such as the number of arrays thereon, the material of construction of the substrate, manufacturing, compatibility with array readers, etc. In general, the substrate is sized to be easily transported. In many embodiments, the substrate will be shaped generally as a rectangular solid, having a length ranging from about 4 mm to about 400 mm, usually from about 4 mm to about 150 mm and more usually from about 4 mm to about 125 mm; a width that ranges from about 4 mm to about 400 mm, usually from about 4 mm to about 120 mm and more usually from about 4 mm to about 80 mm; and a thickness that ranges from about 0.01 mm to about 5.0 mm, usually from about 0.1 mm to about 2 mm and more usually from about 0.2 mm to about 1 mm. For example, where the substrate is rectangularly shaped and is configured to carry about from about 1 to about 200 sample evaluation arrays thereon, e.g., a glass, rectangular substrate or the like, and which is compatible with an Agilent MICROARRAY SCANNER available from Agilent Technologies, Palo Alto, Calif. the dimensions of the substrate may be about 1″×3″×1 mm. Such dimensions are exemplary only and are in no way intended to limit the scope of the invention.

Immobilization of the probes to the substrate may be performed using conventional techniques. See, e.g., Letsinger et al. (1975) Nucl. Acids Res. 2:773-786; Pease, A. C. et al., Proc. Nat. Acad. Sci. USA, 1994, 91:5022-5026 and AOligonucleotide Synthesis, a Practical Approach, @ Gait, M. J. (ed.), Oxford, England: IRL Press (1984). The surface of a substrate may be treated with an organosilane coupling agent to functionalize the surface. See, e.g., Arkins, ASilane Coupling Agent Chemistry,@ Petrarch Systems Register and Review, Eds. Anderson et al. (1987).

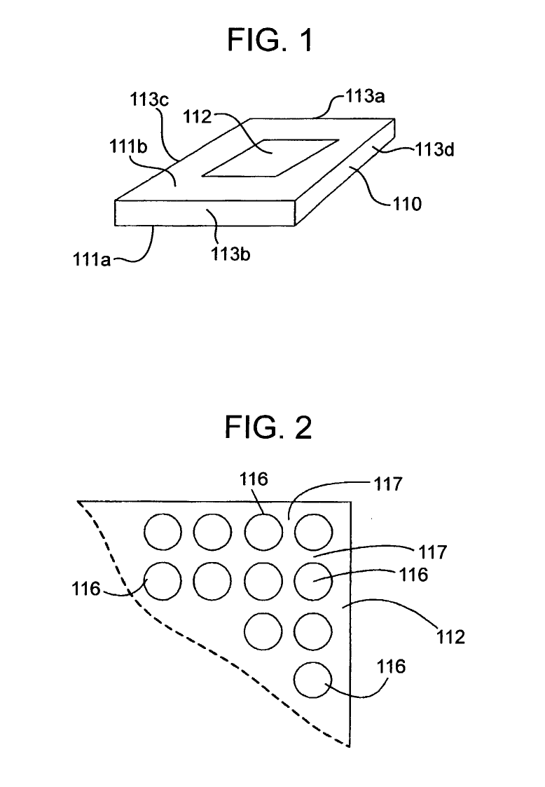

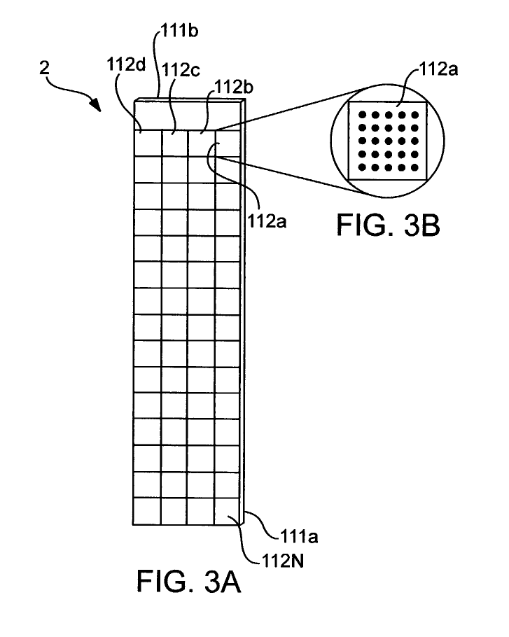

Referring first to FIGS. 1-3, where like numerals represent like features or components, FIG. 1 shows an exemplary embodiment of a sample evaluation device 1 of the present invention. Sample evaluation device 1 includes a contiguous planar substrate 110 carrying sample evaluation array 112, as described above, disposed on a rear surface 111b of substrate 110. It will be appreciated though, that more than one sample evaluation array (any of which are the same or different) may be present on rear surface 111b, with or without spacing between such sample evaluation arrays, as will be described in greater detail below (see FIGS. 3A and 3B). A front surface 111a of the slide 110 usually does not carry any sample evaluation arrays 112. As mentioned above, sample evaluation array 112 contains multiple spots or features 116 of probes, e.g., in the form of olignucleotides or polynucleotides, as is shown in FIG. 2 which shows an enlarged view of a portion of device 1 showing spots or features 116.

As mentioned above, all of the features 116 may be different, or some or all could be the same. As described above, each array is configured to correspond to low and high expressed genes in a broad range of tissues, cell types and species. The interfeature areas 117, if present, could be of various sizes and configurations. As described above, each spot or feature carries a predetermined probe such as a predetermined oligonucleotide or polynucleotide (which includes the possibility of mixtures of oligonucleotides or polynucleotides) and usually multiple copies of such predetermined probe molecule. It will be understood that there may be a linker molecule (not shown) of any known types between the rear surface 111b and the first nucleotide.

A feature of the subject invention is that the sample evaluation arrays are configured to evaluate a sample of at least one detectably labeled target using a small amount of the sample (an amount that does not exceed about 5 μl may be used where an amount of sample as little as about 1 μl may be used in certain embodiments, as will be described in greater detail below). Accordingly, a subject sample evaluation array is sized such that a small amount of sample exposed to a subject sample evaluation array will contact each feature of the sample evaluation array such that each feature, and thus each probe, is exposed to a sufficient amount of sample, i.e., each spot is exposed to enough sample to enable binding of target in the sample to the sample evaluation array if the target and the probe are complementary.

FIG. 3A shows an exemplary embodiment of sample evaluation device 2 having a plurality of sample evaluation arrays 112a-112N thereon, where “N” is an integer corresponding to the number of arrays present. Each sample evaluation array 112a-112N is configured as multiple features or spots of probes, as described above and as shown in the enlarged view of array 112a in FIG. 3A. As shown, each array is segregated or separated from every other array, for example by a physical barrier, hydrophobic strip or by being sufficiently spaced-apart from other arrays so that sample does not spread to other arrays. As mentioned above, each sample evaluation array 112a-112N may be the same or a number of the arrays may be the same, for example to test a plurality of samples against the same sample evaluation array at the same time and under the exact same conditions without cross-contamination, or one or more arrays may be different, for example to test the same sample against different arrays at the same time and under the exact same conditions without cross-contamination.

Methods

The subject invention also provides methods for evaluating whether a sample of at least one detectably labeled target biomolecule is suitable for use in a biopolymeric array assay. That is, the subject methods are employed to evaluate the quality of a detectably labeled sample, i.e., to evaluate the performance of the sample, before the sample is used in an array assay such as a hybridization assay or the like, thereby saving time and expense by determining how the sample is going to perform in an array assay before the sample is used in the array assay.

Generally, the subject methods include providing a substrate having at least one sample evaluation array thereon, i.e., a subject sample evaluation device as described above, contacting the at least one sample evaluation array with a sample of at least one labeled target biomolecule under suitable hybridization conditions, detecting any resultant surface bound duplex nucleic acids to obtain signal data and processing the signal data to evaluate whether the sample is of sufficient quality for use in an array assay such as a hybridization assay. Embodiments of the subject methods include using only a relatively small or minimal sample amount for sample evaluation and/or incubating the array with the sample being evaluated for a minimal or relatively short period of time. The subject methods also advantageously enable a plurality of samples to be evaluated at the same time under the same conditions using a single sample evaluation device, e.g., the same sample may be evaluated with a plurality of different arrays (or the same arrays, e.g., for quality control purposes) at the same time under the same conditions without cross-contamination or a plurality of different samples may be evaluated with a plurality of the same or different arrays at the same time under the same conditions without cross-contamination.

In practice, the sample evaluation array is contacted with a fluid sample containing at least one labeled target biomolecule. The target molecule may be mRNA that is labeled and used directly as the target, or, typically, the target is cRNA or cDNA that is representative of mRNA from a cell or tissue, where the at least one detectably labeled target is complementary to at least one probe sequence attached to the array surface. The sample may be introduced to the sample evaluation array using a pipette, syringe, dipping the array into the sample, or any other suitable introduction protocol, where the sample may be any suitable sample which includes at least one target, i.e., at least one member of a specific binding pair. That is, the sample will include targets that are capable of binding with a complementary probe bound to the surface of the substrate.

The sample is contacted with the sample evaluation array under conditions that promote or provide for the binding of detectably labeled targets in the sample to the sample evaluation array, i.e., to their complementary probe on the substrate surface, to form a binding complex on the substrate surface, i.e., an interaction between a target and a probe. Accordingly, a binding complex is formed at those locations on the array where a probe is present that is the complement of a target in the sample, where such complex may be detected due to the label of the target. The conditions employed are such that specific binding is obtained using the subject invention, e.g., under shortened hybridization time and/or minimal sample volume, with minimal non-specific binding to the probes on the substrate surface or to the substrate surface itself

For hybridization assays, the array may be contacted with the sample under stringent hybridization conditions that are compatible with the formation of duplexes on the array surface between complementary nucleic acid probes and their corresponding complementary nucleic acid targets that are present in the sample, e.g., their corresponding mRNA targets present in the sample. An example of stringent hybridization conditions is hybridization at 60° C. or higher and 3×SSC (450 mM sodium chloride/45 mM sodium citrate). Another example of stringent hybridization conditions is incubation at 42° C. in a solution containing 30% formamide, 1M NaCl, 0.5% sodium sarcosine, 50 mM MES, pH 6.5. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate. Alternative or less stringent conditions, such as moderately stringent conditions, may be employed.

As described above, certain embodiments of the subject sample evaluation devices include more than one sample evaluation array, where any of the arrays may be the same or different. Accordingly, the subject methods include applying sample to each sample evaluation array, respectively, where the sample may be the same or different as sample applied to any other array. In this manner, a plurality of sample evaluations may be performed at the same time, under the same conditions using the same device.

In order to detect the bound duplexes on the surfaces of the substrate, at some step prior to the contacting step the targets, e.g., cDNA or cRNA targets, are labeled with a detectable label. Generally, such detectable labels include, but are not limited to, radioactive isotopes, fluorescers, chemiluminescers, enzymes, enzyme substrates, enzyme cofactors, enzyme inhibitors, dyes, metal ions, metal sols, ligands (e.g., biotin or haptens) and the like. In some embodiments of the subject methods, the sample targets e.g., nucleic acids, are directly labeled with a detectable label, wherein the label may be covalently or non-covalently attached to the nucleic acids of the sample. For example, the nucleic acids, including the target nucleotide sequence, may be labeled with biotin, exposed to hybridization conditions, wherein the labeled target nucleotide sequence binds to an avidin-label or an avidin-generating species. In an alternative embodiment, the target analyte such as the target nucleotide sequence is indirectly labeled with a detectable label, wherein the label may be covalently or non-covalently attached to the target nucleotide sequence. For example, the label may be non-covalently attached to a linker group, which in turn is (i) covalently attached to the target nucleotide sequence, or (ii) includes a sequence which is complementary to the target nucleotide sequence. In another example, the probes may be extended, after hybridization, using chain-extension technology or sandwich-assay technology to generate a detectable signal (see, e.g., U.S. Pat. No. 5,200,314).

In one embodiment, the label is a fluorescent compound, i.e., capable of emitting radiation (visible or invisible) upon stimulation by radiation of a wavelength different from that of the emitted radiation, or through other manners of excitation, e.g. chemical or non-radiative energy transfer. The label may be a fluorescent dye. Usually, a target with a fluorescent label includes a fluorescent group covalently attached to a target molecule, e.g., a target nucleic acid, capable of binding specifically to the complementary probe, e.g., complementary nucleic acid sequence.

A feature of the subject methods is that only a small amount of sample is needed to evaluate whether the sample is suitable for use in an array assay. In this manner, a sufficient amount of sample is conserved for use in the array assay. Typically, less than about 1/20 to about ⅕ of the total sample amount is used for sample evaluation according to the subject invention. In certain embodiments, a volume of sample that ranges from about 1 μl to about 5 μl, usually from about 1 μl to about 3 μl, is contacted with each sample evaluation array. In using such a limited amount of sample, the amount of sample is not significantly depleted during the evaluation thereof, thereby conserving a sufficient amount for use in a subsequent array assay, that is if the quality of the sample is determined suitable for use in an array assay. Furthermore, such a small sample amount enables multiple samples to be contacted with multiple arrays on a substrate without barriers therebetween to prevent cross-contamination because the amount of sample spreading or sample diffusion across the surface of the substrate is minimal due to the small size of the sample droplet applied and surface tension of the droplet. Still further, such a small sample volume enables sample evaluation to be preformed without the use of a cover over the sample evaluation array to contain the sample on the array.

The sample evaluation array is then incubated with the sample of at least one labeled target under appropriate hybridization conditions, as mentioned above, where conditions may vary depending on the particular array and binding pair. A feature of the subject invention is that the incubation period between the sample and the sample evaluation array is minimal, typically the incubation period does not exceed about 4 hours, where usually the incubation period ranges from about 30 minutes to about 4 hours and more usually from about 1 hour to about 3 hours. The sample evaluation device is typically incubated in a humidified chamber, where the humidity is maintained in a range from about 70% to about 90%. The subject methods are typically carried out in a moderate humidity environment. By “moderate humidity” is meant an environment in which the humidity is at least about 70% relative humidity, usually at least about 85% relative humidity and more usually at least about 90% relative humidity. Alternatively or in addition, one may apply an evaporation retarding agent, e.g. mineral oil, glycerol solution, polyethylene glycol, etc., over the surface of the deposited sample.

Once the incubation step is complete, the sample evaluation array is washed at least one time to remove any unbound and non-specifically bound sample from the substrate, generally at least two wash cycles are used. Washing agents used in array assays are known in the art and, of course, may vary depending on the particular binding pair used in the particular assay. For example, in those embodiments employing nucleic acid hybridization, washing agents of interest include, but are not limited to, salt solutions such as sodium, sodium phosphate (SSP) and sodium, sodium chloride (SSC) and the like as is known in the art, at different concentrations and which may include some surfactant as well.

Following the washing procedure, the sample evaluation array is then interrogated or read to detect any resultant surface bound duplex nucleic acids or binding complexes to obtain signal data related to the presence of the surface bound duplex nucleic acids, i.e., the label is detected using calorimetric, fluorimetric, chemiluminescent, bioluminescent means or other appropriate means.

Reading of the sample evaluation array to obtain signal data may be accomplished by illuminating the array and reading the location and intensity of resulting fluorescence at each feature of the array to obtain a result. For example, an array scanner may be used for this purpose that is similar to the Agilent MICROARRAY SCANNER available from Agilent Technologies, Palo Alto, Calif. Other suitable apparatus and methods for reading an array to obtain signal data are described in U.S. patent application Ser. No. 09/846,125 “Reading Multi-Featured Arrays” by Dorsel et al.; and Ser. No. 09/430,214 “Interrogating Multi-Featured Arrays” by Dorsel et al., the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583, the disclosure of which is herein incorporated by reference, and elsewhere).

The obtained signal data from the reading may be in any convenient form, i.e., may be in raw form or may be in a processed form. Regardless, the obtained signal data is processed to evaluate whether the sample is suitable for use in a subsequent array assay. Such processing may be performed manually or automatically, e.g., by a data processor, e.g., a microprocessor operatively associated with the array reader, a personal computer (PC), or the like.

The signal data provided from the reading of these arrays may be analyzed in a variety of manners in order to determine the quality of the sample contacted with an array. For example, relative signal intensities and graphical representations thereof may be used to evaluate sample quality. That is, signal data may be evaluated based on the intensity of signal of the spots or features of the array, i.e., the probes, and the intensity of signal around the spots, i.e., background or non-probe regions. For example, a sample may be evaluated as suitable for use in an array assay if the signal intensity of the spots yields sufficiently high signal to noise, e.g., above a certain threshold level, and the background signal is low, e.g., below a certain threshold level, indicating that the at least one target in the sample is sufficiently labeled with a detectable label, while at the same time no, or an insignificantly small amount of, “free” label in sample is present, i.e., signal arising from the label itself when it is not attached to a target. A ratio of probe feature signal intensity to non-probe region or negative control or reverse complement probe signal intensity may also be employed to determined sample quality, where, for example, a ratio above a predetermined ratio may indicate suitability of sample for use in an array. For example, a ratio of binding or evaluation probe signal intensity to the reverse complement signal intensity of about 8-12 may indicate a suitable sample for use in an array assay. By sufficiently labeled is meant that the one or more targets in the sample are labeled to a degree that enables detection using the subject methods. By insignificantly small amount of free label is meant that the level of unincorporated label bound to the substrate surface is not so great to obscure the detection of bound detectably labeled target or otherwise cause erroneous results, of the subject methods.

Other methods of analysis may also be used in accordance with the subject methods. For example, a target may be determined suitable for use in an array assay if, for a particular gene, about the same fluorescent intensity or a consistent fluorescent intensity, is observed across the probe set complementary to that gene, i.e., each probe of the set produces a signal intensity that does not differ by a predetermined amount from any other probe of the set. Likewise, a consistent log ratio across a probe set for each gene may indicate a suitable sample.

In certain embodiments, the signal intensity of each probe, or the average signal intensity of the probes, of a probe set may be compared to a predetermined value such that if the probes provide signal intensity above that predetermined value, the sample is determined to be suitable for use in an array assay. For example, a sample may be determined suitable if about 80% of the binding or evaluation probes provide signal intensities at or above a predetermined signal intensity such as at or above 1000 fluorescent units. In some embodiments, as mentioned above, the sample evaluation arrays include controls such as non-coding probes or probes to the reverse complements of the evaluation probes, i.e., non-binding probes. Accordingly, a sample may be determined suitable if, in addition to the above, less than about 80% of the control or reverse complement probes provides signal intensities less than a predetermined signal intensity such as less than about 1000 fluorescent units.

The results of the sample evaluation reading (processed or not) may be forwarded (such as by communication) to a remote location if desired, and received there for further use (such as further processing). By “remote location” is meant a location other than the location at which the sample evaluation device is present and sample evaluation occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Once a sample is evaluated and determined to be suitable for use in an array assay, the sample may then be used in an array assay. Array assays between surface bound binding agents or probes and target molecules in solution may be used to detect the presence of particular targets in a sample solution. The surface-bound probes may be oligonucleotides, peptides, polypeptides, proteins, antibodies or other molecules capable of binding with target biomolecules in the solution. Such binding assays are used in a variety of different fields, e.g., genomics (in sequencing by hybridization, SNP detection, differential gene expression analysis, identification of novel genes, gene mapping, finger printing, etc.) and proteomics. These arrays include a plurality of ligands or molecules or probes (i.e., binding agents) deposited onto the surface of a substrate in the form of an “array” or pattern.

Specific array assays of interest include hybridization assays in which the evaluated samples are employed. In these assays, a sample of target nucleic acids is contacted with an array of complementary nucleic acid probe sequences under hybridization conditions, whereby complexes are formed between target nucleic acids that are complementary to probe sequences attached to the array surface. The presence of hybridized complexes is then detected. Specific hybridization assays of interest include: gene discovery assays, differential gene expression analysis assays; nucleic acid sequencing assays, and the like. Patents describing methods of using arrays in various applications include U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Other array assays of interest include those where the arrays are arrays of polypeptide binding agents, e.g., protein arrays, where specific applications of interest include analyte detection/proteomics applications, including those described in U.S. Pat. Nos. 4,591,570; 5,171,695; 5,436,170; 5,486,452; 5,532,128; and 6,197,599; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

Generally, an array used in an array assay typically includes at least two distinct polymers that differ by monomeric sequence covalently attached to different and known locations on the substrate surface. Each distinct polymeric sequence of the array is typically present as a composition of multiple copies of the polymer on a substrate surface, e.g., as a spot on the surface of the substrate. The number of distinct polymeric sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like. A variety of array structures/formats (e.g., substrate format, dimensions, materials, nature of probe attachment, probe pattern layout, etc.) are known to those of skill in the art, where representative array structures that may be used with the evaluated samples include those disclosed or referenced in: U.S. Pat. Nos. 6,180,351; 6,232,072; 6,300,137; 6,255,053; 6,428,957; 6,399,394; 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,624,711; 5,639,603; 5,658,734; the disclosures of which are herein incorporated by reference, as well as in WO 93/17126; WO 95/11995; WO 95/35505; EP 742 287; and EP 799 897.

In the broadest sense, such arrays used in array based assays are arrays of polymeric or biopolymeric ligands or molecules, i.e., binding agents, where the polymeric binding agents may be any of: peptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like.

The arrays used in array based assays may be produced by a number of different methods, where such methods are known in the art (see, for example, Khrapko et al., DNA Sequence (1991) 1:375-388; WO 95/35505; U.S. Pat. No. 5,143,854; and Fodor et al., Science (1991) 251:767-773, the disclosures of which are herein incorporated by reference.

As mentioned above, the array used in the array assay contains multiple spots or features of biopolymers, e.g., in the form of polynucleotides, where all of the features may be different, or some or all could be the same. Each feature carries a predetermined biopolymer such as a predetermined polynucleotide (which includes the possibility of mixtures of polynucleotides). It will be understood that there may be a linker molecule (not shown) of any known types between the surface of the substrate and the first nucleotide of a probe.

In using the arrays with a sample that has been evaluated according to the subject invention, as described above, the evaluated sample containing at least one detectably labeled target is contacted with an array such that the at least one target in the sample binds or associates with a complementary probe on the substrate surface to form a binding complex or nucleic acid duplex, i.e., an interaction between a target in the sample and a probe of the array is formed. In other words, the evaluated sample is contacted with the array under conditions that produce duplex nucleic acid structures at those locations on the array where a probe is present that is the complement of a nucleic acid target in the sample, e.g., an mRNA of the sample. Typically, the array surface is contacted with the sample under stringent hybridization conditions that are compatible with the particular probe and target, i.e., binding members, to produce duplexes on the array surface between complementary probes and their corresponding nucleic acid targets that are present in the sample, e.g., their corresponding mRNA targets present in the sample. An example of stringent hybridization conditions is hybridization at 60° C. or higher and 3×SSC (450 mM sodium chloride/45 mM sodium citrate). Another example of stringent hybridization conditions is incubation at 42° C. in a solution containing 30% formamide, 1M NaCl, 0.5% sodium sarcosine, 50 mM MES, pH 6.5. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

The array is then washed and read to detect the presence and location of the bound duplex nucleic acid structures, i.e., the binding complexes. Where the labeled target nucleic acids are fluorescently labeled, reading of the array may be accomplished by illuminating the array and reading the location and intensity of resulting fluorescence at each feature of the array to detect any binding complexes on the surface of the array. For example, a scanner may be used for this purpose such as the AGILENT MICROARRAY SCANNER available from Agilent Technologies, Palo Alto, Calif. Other suitable apparatus and methods are described in U.S. patent applications Ser. No. 09/846,125 “Reading Multi-Featured Arrays” by Dorsel et al.; and Ser. No. 09/430,214 “Interrogating Multi-Featured Arrays” by Dorsel et al. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample).

Once the results, i.e., assay data, are obtained, the results are employed to determine the presence of the nucleic acid targets in the assay sample. In other words, the presence of the target(s) in the sample is then deduced from the detection of labeled target nucleic acids on the substrate surface, where the location of a given labeled target nucleic acid imparts information about the identity of the corresponding nucleic acid analyte and the intensity of the signal may impart information regarding the quantity of the corresponding nucleic acid analyte in the sample.

In certain embodiments, the subject methods include a step of transmitting data from at least one of the detecting and deriving steps, as described above, to a remote location. By “remote location” it is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Kits

Finally, kits for evaluating a sample as suitable for use in a biopolymeric array are provided. The subject kits at least include one or more subject sample evaluation devices, where the devices may include one or more sample evaluation arrays. Typically, a plurality of subject sample evaluation devices is included. The kits may also include one or more components for preparing sample and/or labeling sample with a detectable label, e.g., sample preparation reagents, labels, buffers and the like. The kit may further include one or more containers such as vials or bottles, with each container containing a separate component for carrying out sample evaluation. The kit may also include a denaturation reagent for denaturing the target, buffers such as hybridization buffers, wash mediums, enzyme substrates, negative and positive controls and written instructions for using the subject sample evaluation devices for carrying out the evaluation of a sample for determining the suitability of the sample for use in an array assay. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. The kit may further include one or more biopolymeric arrays for performing an array assay with an evaluated sample.

### EXPERIMENTAL

The following examples are put forth so as to provide those of ordinary skill in the art with a complete disclosure and description of how to make and use the present invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The following examples describe the use of sample evaluation arrays in accordance with the subject invention and analysis of the reading thereof after contact with a labeled sample. As further described below, a determination of whether a sample contacted with a subject array is suitable for use in an array assay may be made based on the analysis of the reading.

### Example I

### Suitable Sample

5 ug of Hela total RNA was converted to Cy3-labeled cDNA using the Agilent Direct Label kit (part no. G2557A) provided by Agilent Technologies, Inc. of Palo Alto, Calif. In a separate reaction, 5 ug of Spleen total RNA was converted to Cy5-labeled cDNA using the Agilent Direct Label kit. The MMLV RT was inactivated by incubation at 65C for 15 min. and the RNA was degraded by adding 0.01 mg RNAse I “A” and incubating at room temperature for 30 minutes. The two reactions were combined and the fluorescently labeled cDNA was purified using the Qiagen PCR purification kit (part no. 28104) available from Qiagen, Inc, of Valencia, Calif. The cDNA was dried in a speed vac and resuspended in 7.5 uL sterile water. The Cy3- and Cy5-labeled cDNA was cohybridized to an array containing 22 genes in which each gene was tiled with 60mer probes every 50 nucleotides covering the entire coding sequence 5′ to the 3′ end of the gene. The reverse complement of every probe (negative probe) was also on the array. The Agilent In Situ Hybridization kit and protocol (part nos. G2559A and G2559-90010) was used for hybridization, available from Agilent Technologies, Inc. of Palo Alto, Calif. The array was scanned on the Agilent MICROARRAY SCANNER available from Agilent Technologies, Palo Alto, Calif. The data was analyzed by plotting fluorescent signal intensity versus gene sequence and log ratio of Cy5/C/3 signal intensities versus gene sequence.

In this example, an array of binding probes complementary to the target strand of the EF-1 gene were prepared by tiling across the gene from the 5′ end to the 3′ end. Negative probes or probes of the reverse complements of each binding probe (probes to the − strand) were also prepared.

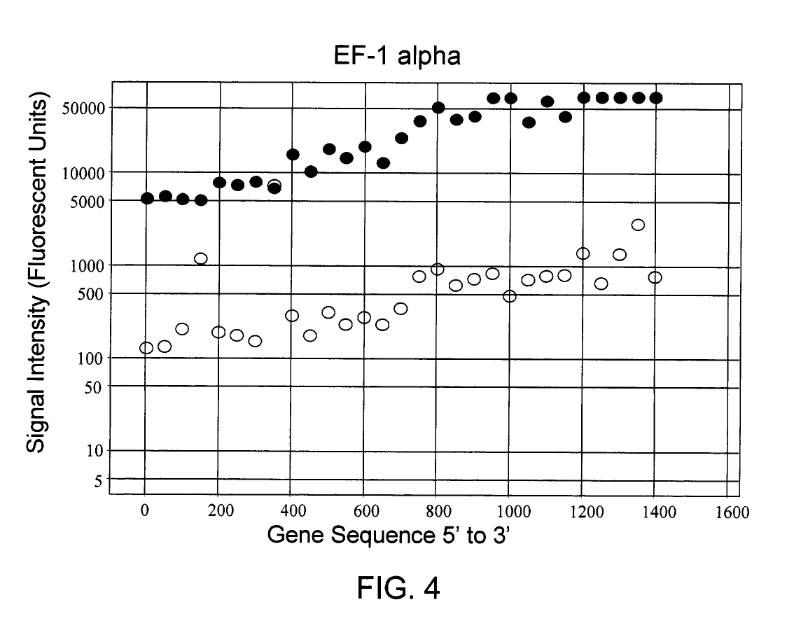

FIG. 4 shows the result of the reading of this binding assay wherein signal intensity (background subtracted fluorescent signal) of each probe versus gene sequence from 5′ to 3′ is plotted on a graph. As shown, the binding probes (closed circles) produce a higher signal intensity compared to the negative probes (open circles). The − strand gives an indication of non-specific binding and background from the sample. Furthermore, the fluorescent signal is constant across the probe set from the 3′ to the 5′ of the gene. These results indicate that the sample is suitable for use in an array assay.

### Example II

### Non-Expressed Gene

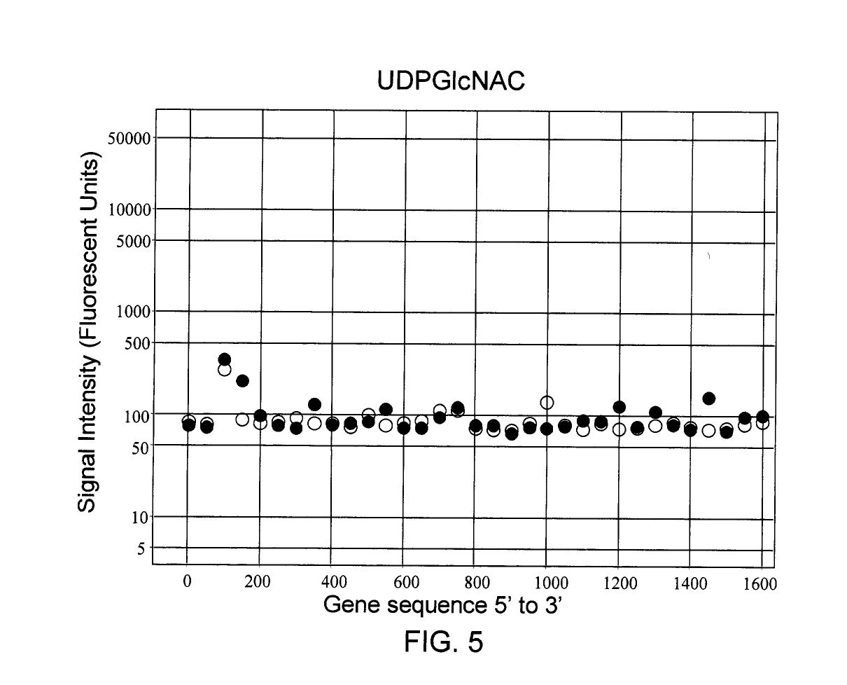

FIG. 5 shows the results of this binding assay for a second gene that is not expressed in the sample. As shown, the signal intensities of each binding probe is substantially similar to that of its reverse complement indicating that the gene is not being expressed in the sample.

### Example III

### Cross Hybridization and Non-Specific Binding

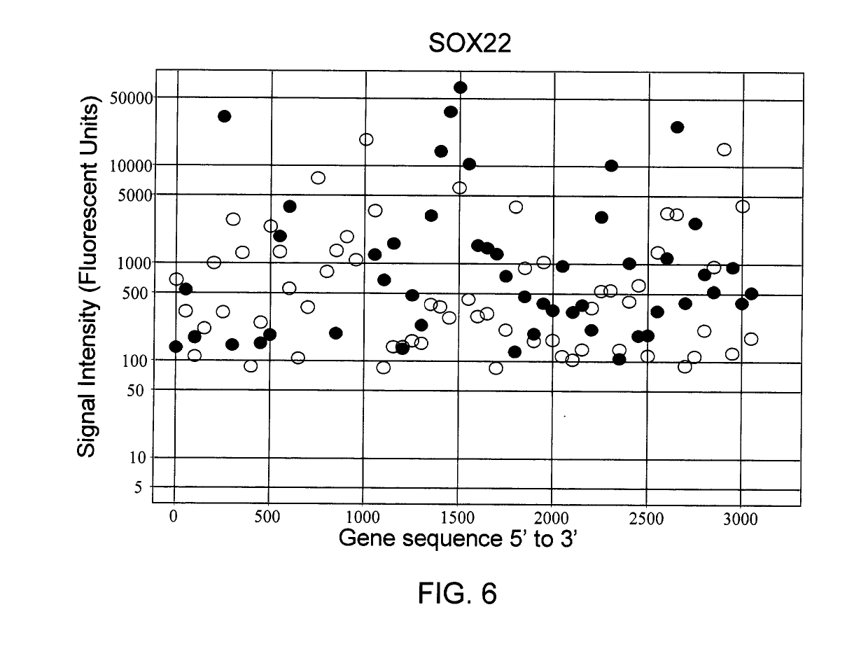

As shown in FIG. 6, both the binding probes and the reverse complement probes produce a random pattern when plotted indicating cross hybridization and non-specific binding.

### Example IV

### Suitable Sample Compared to Unsuitable Sample

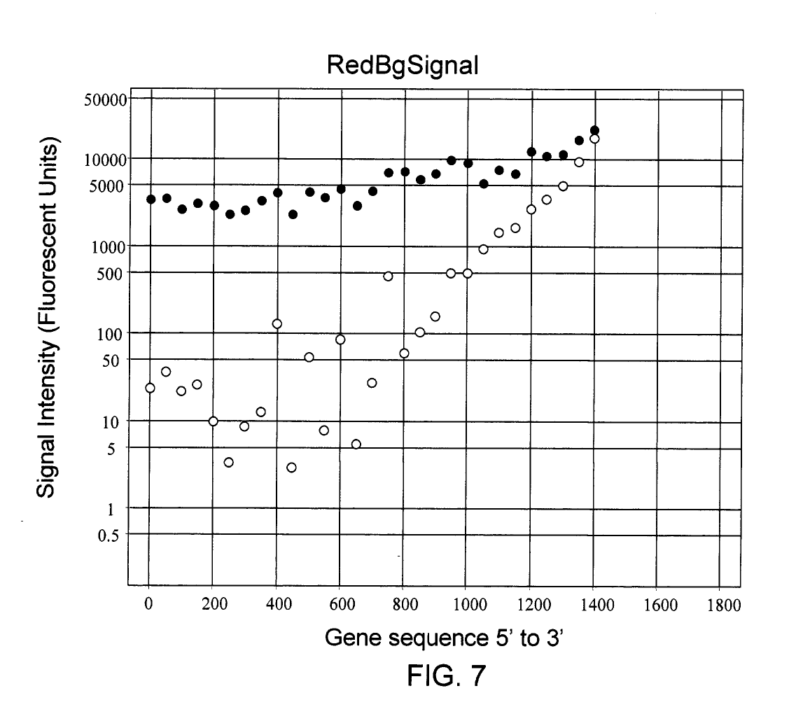

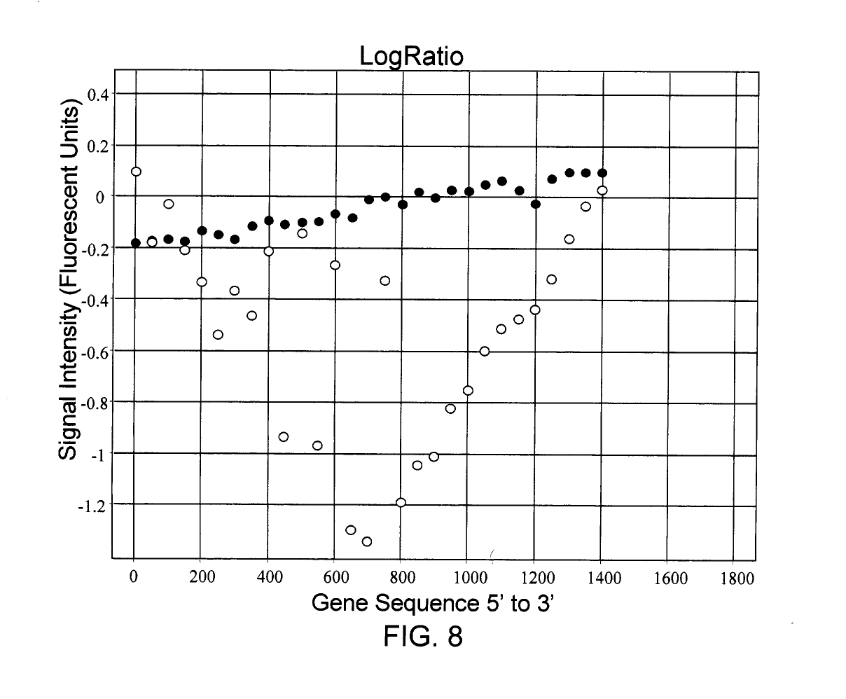

FIG. 7 shows the result of this binding assay. The darkened circles indicate a suitable sample as evidenced by the constant fluorescent signal across the probes. However, the lighter colored circles indicate a sample that is not suitable for use in an array assay as evidenced by the dramatic fall-off in signal moving away from the 3′ end. FIG. 8 shows the log ratio of the Cy5- and Cy3-samples. Again, the darkened circles indicate a suitable sample, as evidenced by the constant log ratio across the probe set. However, the lighter colored circles indicate a sample that is not suitable for use in an array assay as evidenced by the dramatic fall-off in log ratio moving away from the 3′ end.

It is evident from the above results and discussion that the above described invention provides devices and methods for evaluating the quality of a sample of labeled target biomolecules prior to use in an array assay. The above described invention provides for a number of advantages, including ease of use, cost effectiveness, short incubation time, use of small sample amounts and the ability to evaluate multiple samples at the same time without cross-contamination. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for evaluating whether a sample of at least one labeled target biomolecule is suitable for use in a biopolymeric array assay, said method comprising:
(a) providing a substrate having an array thereon;
(b) contacting said array with a volume that does not exceed about 5 μl of said sample;
(c) detecting any resultant surface bound target biomolecules to obtain signal data; and
(d) processing said signal data to evaluate whether said sample of at least one detectably labeled target biomolecule is suitable for use in a biopolymeric array assay.

2. The method according to claim 1, wherein said sample volume ranges from about 1 μl to about 5 μl.

3. The method according to claim 1, wherein said conditions comprise incubating said array with said sample for a period of time that does not exceed about 4 hours.

4. The method according to claim 3, wherein said period of time ranges from about 1 hour to about 3 hours.

5. The method according to claim 1, wherein said at least one target biomolecule is a nucleic acid.

6. The method according to claim 1, wherein said substrate comprises a plurality of arrays and said method further comprises contacting each of said plurality of arrays with a sample comprising at least one labeled target biomolecule to simultaneously evaluate each sample of said plurality of samples.

7. The method according to claim 6, wherein at least two of said plurality of arrays are different.

8. The method according to claim 6, wherein each of said plurality of samples is the same.

9. The method according to claim 6, wherein at least two samples of said plurality of samples are different.

10. The method according to claim 1, wherein said at least one labeled target biomolecule is a fluorescently labeled target biomolecule.

11. A method for evaluating the quality a sample of at least one detectably-labeled target biomolecule as suitable for use in a biopolymeric array assay, said method comprising:
(a) providing a substrate having an array thereon;
(b) contacting said array with said sample;
(c) incubating said array and said sample for a period of time that does not exceed about 4 hours;
(d) detecting any resultant surface bound detectably-labeled target biomolecules to obtain signal data; and
(e) processing said signal data to evaluate whether said sample of at least one detectably labeled target biomolecule is suitable for use in a biopolymeric array assay.

12. The method according to claim 11, wherein said period of time ranges from about 1 hour to about 3 hours.

13. The method according to claim 11, wherein a quantity of said sample that does not exceed about 5 μl is contacted to said array.

14. The method according to claim 13, wherein said quantity ranges in size from about 1 μl to about 5 μl.

15. The method according to claim 11, wherein said at least one labeled target biomolecule is a nucleic acid.

16. The method according to claim 11, wherein said substrate comprises a plurality of arrays and said method further comprises contacting each of said plurality of arrays with a plurality of samples, respectively, each sample comprising at least one detectably-labeled target biomolecule, to simultaneously evaluate each sample of said plurality of samples.

17. The method according to claim 16, wherein at least two of said plurality of arrays are different.

18. The method according to claim 16, wherein each of said plurality of samples is the same.

19. The method according to claim 16, wherein at least two samples of said plurality of samples are different.

20. The method according to claim 11, wherein said at least one labeled target biomolecule is a fluorescently labeled target biomolecule.

21. A method of performing a biopolymeric array assay, said method comprising:
(a) evaluating whether a sample of at least one detectably labeled target biomolecule is suitable for use in an array assay according to the method of claim 1; and
(b) performing an array assay with said evaluated sample.

22. The method according to claim 21, further comprising reading the result of said array assay.

23. A method comprising forwarding data representing a result of a reading obtained by the method of claim 21.

24. The method according to claim 23, wherein said data is transmitted to a remote location.

25. A method comprising receiving data representing a result of a reading obtained by the method of claim 21.

26. A device for evaluating the quality of a sample of at least one labeled target biomolecule as suitable for use in a biopolymeric array assay, said device comprising:
a substrate having at least one evaluation array thereon, wherein said at least one evaluation array is configured to evaluate said sample using a volume of said sample that does not exceed about 5 μl.

27. The device according to claim 26, wherein said evaluation array comprises from about 4 to about 1000 features of probe molecules.

28. The device according to claim 27, wherein about 1,000 to about 100,000 probe molecules are present in each feature.

29. The device according to claim 27, wherein at least some of said features comprise probes of repetitive sequences.

30. The device according to claim 26, wherein at lease one evaluation array comprises tiled probes.

31. The device according to claim 26, wherein said substrate comprises a plurality of evaluation arrays.

32. The device according to claim 31, wherein at least two of said plurality of evaluation arrays are different.

33. The device according to claim 26, wherein said at least one evaluation array includes probes to relatively well-conserved genes between at least two species.

34. A kit for evaluating the quality of a sample of at least one labeled target biomolecule as suitable for use in a biopolymeric array assay, said kit comprising:
(a) at least one array; and
(b) instructions for using said at least one evaluation array to evaluate the whether a sample is suitable for use in a biopolymeric array assay according to the method of claim 1.

35. The kit according to claim 34, further including components for labeling said sample with a detectable label.
